(19) United States
(12) Patent Application Publication
(10) Pub. No.: US 2005/0261565 A1
(43) Pub. Date: Nov. 24, 2005

(54) Discretely coated sensor for use in medical electrodes

(75) Inventors: Frederick W. Lane, Leominster, MA (US); Peter A. Pignone, Framingham, MA (US)

Correspondence Address: FITCH EVEN TABIN AND FLANNERY, 120 SOUTH LA SALLE STREET, SUITE 1600, CHICAGO, IL, 60603-3406, US

(73) Assignee: MICRON MEDICAL PRODUCTS

(21) Appl. No.: 10/848,488

(22) Filed: May 18, 2004

(51) Int. Cl. A61B 5/04

(52) U.S. Cl. 600/394; 600/395; 600/396

(57) ABSTRACT

Discretely coated sensors or eyelets are provided for use in medical electrodes. Also provided are sensors for use in medical electrodes where the sensors are coated with a with a silver/silver chloride coating in a discrete area. Silver/silver chloride medical electrodes are provided that have a sensor or eyelet made of conductive plastic which is discretely coated with silver/silver chloride only in the area in which the sensor makes direct contact with the electrolyte gel in the conductive pathway.

### FIELD OF THE INVENTION

The present invention relates to discretely coated sensors or eyelets for use in medical electrodes. More particularly, this invention relates to sensors for use in medical electrodes where the sensor is coated with a silver/silver chloride coating in a discrete area. This invention also relates to methods for making and using such discretely coated electrodes.

### BACKGROUND OF THE INVENTION

Medical electrodes are used in many different applications for a variety of purposes. Monitoring electrodes and diagnostic electrodes are used to detect electrical activity from a patient's body. TENS electrodes are used to provide electrical stimulation to a patient's body.

One type of commonly used electrode is a silver/silver chloride (Ag/AgCl) electrode. Silver/silver chloride electrodes are generally used in biofeedback (e.g., ECG, EEG, and apnea) and bio-stimulation (e.g., TENS, EMS) products. Silver/silver chloride electrodes include a sensor or eyelet which has a surface coating of a conductive metal, silver, interfaced with its salt, silver chloride. One surface of the sensor or eyelet is coupled to a patient's body through an appropriate electrolyte gel. Another surface of the eyelet is mated to a conductive snap. The conductive snap is used to interconnect the patient, through the eyelet and electrolyte, to an ECG or similar recording apparatus or to an electrical stimulator.

The prior art silver/silver chloride sensors or eyelets are generally made of a non-conductive plastic which is coated with silver/silver chloride. Electrode materials such as silver/silver chloride do not allow significant buildup of offset potentials. Silver/silver chloride electrodes have been shown to possess superior characteristics to that of unchlorided silver electrodes when used for recording low level AC and DC potentials. Chlorided silver electrodes generally present less low frequency “noise” than either gold or silver electrodes.

Traditionally, silver/silver chloride electrode systems utilize sensors that are completely coated with silver/silver chloride. The conductive snap is generally made of stainless steel, nickel coated brass, or a conductive resin. Silver/silver chloride electrodes produce lower and more stable junction potentials than many other electrode designs. However, the presence of dissimilar electrolytic interfaces still results in junction potentials and can cause electrode based artifacts. Because sensors are traditionally fully coated with silver/silver chloride, the silver/silver chloride post of the eyelet is in contact with the metal snap. The contact of dissimilar metals (i.e., the silver/silver chloride coated post of the eyelet and the conductive snap) creates a voltage difference. This bimetallic potential can lead to instability and a degrading of electrical performance. This issue can be aggravated further if there is a leak of electrolyte into the junction of the silver/silver chloride post of the eyelet and the conductive metal stud of the snap, thereby causing corrosion and possibly leading to serious offset potential drift and erratic signals.

It would be desirable to provide an improved silver/silver chloride electrode system that minimizes the use of silver—a heavy metal. It would also be desirable to provide a silver/silver chloride electrode system which eliminates the problem of having two dissimilar metals in direct contact, and thereby reduces or eliminates the instability and possible failure of the electrode.

### SUMMARY OF THE INVENTION

This invention relates to sensors having a conductive metallic coating in a discrete area for use in medical electrodes. More specifically, the invention provides sensors having a silver/silver chloride coating in a discrete area for use in medical electrodes. The invention provides a silver/silver chloride medical electrode having a sensor or eyelet made of conductive plastic which is discretely coated with silver/silver chloride only in the area in which the sensor makes direct contact with the electrolyte gel in the conductive pathway. The post portion of the sensor comprises un-coated, non-metallic conductive base material and provides the necessary conductive path between the silver/silver chloride surface and the conductive snap.

The discretely coated electrode of the present invention provides an electrode system that avoids the direct contact of two dissimilar metals, and allows the electrode to meet the established requirements for such products while reducing or eliminating the potential of instability and/or failure of the finished product. Additionally, the elimination of the dissimilar metal problem allows for the use of more reactive metals, such as brass, for the snap which is intended for connection to the post of the eyelet.

The present invention provides a discretely coated conductive plastic eyelet for use in a medical electrode, the eyelet comprising a base portion having a bottom surface and a top surface, and a post portion integral with and extending upwardly from the top surface of the base portion, wherein the bottom surface of the base portion is at least partially coated with a conductive material.

The present invention further provides a discretely coated eyelet for use in a medical electrode wherein the eyelet comprises a plastic resin loaded with carbon fiber. The present invention also provides a discretely coated eyelet for use in a medical electrode wherein the bottom surface of the eyelet, which comes in contact with the electrolyte, is at least partially coated with silver/silver chloride.

In one aspect, the present invention provides a discretely coated sensor for a silver/silver chloride medical electrode that eliminates the dissimilar metal phenomenon created in standard metal snap electrode designs. In another aspect, the present invention provides a discretely coated sensor for a silver/silver chloride medical electrode in which only a pre-determined portion of the eyelet is coated with silver/silver chloride.

In another aspect, the present invention provides a discretely coated sensor for a silver/silver chloride electrode which minimizes the amount of heavy metal introduced into the environment when disposable silver/silver chloride electrodes are discarded.

In another aspect, the present invention provides a discretely coated sensor for a silver/silver chloride electrode that can meet established industry requirements for physiological monitoring while providing a stable conductive interface when mated with metal snaps.

In another aspect, the present invention provides a discretely coated sensor for a silver/silver chloride electrode that has an increased shelf life due to improvement in the stability of the conductive plastic and conductive metal connector interface.

In another aspect, the present invention provides a discretely coated sensor or eyelet that is “universal,” i.e;, can be used with a snap or connector made of any type of material commonly used.

In another aspect, the present invention provides a discretely coated sensor for a silver/silver chloride electrode system that allows the use of naturally conductive metal in the snap without the need to coat or plate with a more resistive coating. For example, the currently used nickel-plated brass snaps can be replaced with brass snaps. In another aspect, the present invention provides a discretely coated sensor for use in a silver/silver chloride electrode system that significantly reduces the use of environmentally hazardous materials in the metal plating processes of the electrode.

These and other features and advantages of the present invention will become apparent to those skilled in the art upon a reading of the following detailed description when taken in conjunction with the drawings wherein preferred embodiments of the invention are shown and described.

### BRIEF DESCRIPTION OF THE DRAWINGS

FIG. 1a is an exploded view of the typical construction of an ECG electrode.

FIG. 1b is a schematic top view of the electrode of FIG. 1a.

FIG. 2a is a schematic top plan view of a discretely coated eyelet for an electrode according to the present invention.

FIG. 2b is a schematic side elevation view of a discretely coated eyelet for an electrode according to the present invention.

FIG. 2c is a schematic perspective view of a discretely coated eyelet for an electrode according to the present invention.

FIG. 3a is a schematic top plan view of a conductive snap for an electrode according to the present invention.

FIG. 3b is a schematic side elevation view of a conductive snap for an electrode according to the present invention.

FIG. 3c is a schematic perspective view of a conductive snap for an electrode according to the present invention.

### DETAILED DESCRIPTION OF THE PREFERRED EMBODIMENTS

The present invention relates to a discretely coated sensor for use in a medical electrode in which the connection between the snap and the sensor or eyelet does not result in the dissimilar metal phenomenon created in standard metal snap electrode designs. By coating the eyelet with a conductive metallic surface only on the area of the eyelet that is in contact with the electrolyte gel, the electrical performance and stability of the electrode are improved.

This invention relates to a medical electrode having a sensor with a silver/silver chloride coating in a discrete area. The invention provides a silver/silver chloride medical electrode system having a sensor or eyelet made of conductive plastic which is discretely coated with silver/silver chloride only in the area in which the sensor makes direct contact with the electrolyte gel in the conductive pathway. The post portion of the sensor comprises un-coated, non-metallic conductive base material and provides the necessary conductive path between the silver/silver chloride surface and the conductive snap. In other words, the portion of the sensor which comes into contact with the electrolyte is coated with silver/silver chloride. The post portion of the sensor that contacts the snap, however, is un-coated. With this construction, the portion of the eyelet that contacts the conductive snap is preferably a conductive carbon loaded plastic material which is not coated and is not metallic.

The discretely coated sensor of the present invention provides an electrode system that avoids the direct contact of two dissimilar metals, and allows the electrode to meet the established requirements for such products while reducing or eliminating a potential of instability and/or failure of the finished product. Additionally, the elimination of the dissimilar metal problem allows for the use of more conductive and less costly metals, such as brass, for the snap which connects to the electrode. The discretely coated sensor or eyelet of the present invention is “universal” in that it can be used with any type of connector or snap. Because the post of the eyelet is not metallic, the eyelet can be coupled with any type of conductive snap (either metallic or non-metallic) while still eliminating the dissimilar metal problem.

The discretely coated sensor of the present invention is a standard shaped eyelet which is formed of a carbon filled thermoplastic resin, such as Acrylonitrile-Butadiene-Styrene (ABS), with inherent conductivity. Silver/silver chloride is deposited on the bottom surface of the eyelet that is in contact with the electrolyte gel, thereby creating a stable interface for monitoring bio-potentials. The post portion of the eyelet is conductive, but uncoated, and makes contact with the snap. With the elimination of the silver/silver chloride at the eyelet/snap interface, a variety of conductive materials can be used for the snap, as the need for a corrosion resistant metal, such as nickel-plated brass or stainless steel, is no longer a significant requirement. Even in the presence of electrolyte contamination of the eyelet/snap interface, the elimination of silver/silver chloride in this area reduces or eliminates corrosion and the resulting bimetallic potentials that can interfere with the performance of the electrode.

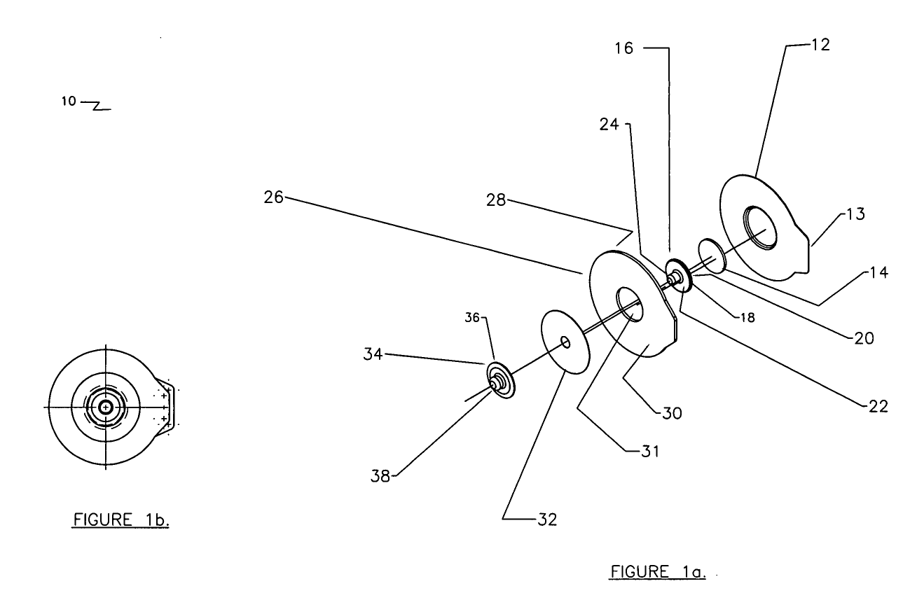

Referring to the drawings, the present invention will be described in the context of a conventional electrode arrangement as shown in FIG. 1. The electrode arrangement discussed herein has been selected for illustration purposes only and is not meant to limit the scope of the invention which is incorporated in this type of electrode. Rather, the discretely coated sensor of the present invention may be used in any of a wide variety of electrode arrangements.

One embodiment of a discretely coated silver/silver chloride electrode 10 is shown in FIG. 1 a. Generally, a sensor or eyelet 16 is snapped together with a conductive snap 34 such that the post portion 24 of the eyelet 16 projects through an aperture 31 in a foam body 26 and into the receiving interior of the stud portion 38 of conductive snap 34. Optionally, a label 32 may be included on the top surface 30 of the foam body 26. The foam body 26 is positioned to surround the conductive snap 34 and the eyelet 16. The foam body 26 includes an adhesive backing 28 on its bottom surface. The aperture 31 in the foam body 26 is sufficiently large so it does not interfere with the direct contact between the conductive snap 34 and the eyelet 16. Preferably, an electrolyte layer 14 completely overlies the bottom surface 20 of the eyelet 16. A release liner 12 is adhered to the adhesive backing 28 of the foam body 26. The release liner 12 preferably includes a tab 13 to facilitate peeling the release liner 12 away from the adhesive backing 28 of the foam body 26.

As shown in FIG. 1a, the electrode is formed by a sensor or eyelet 16 and a conductive snap 34. In one embodiment, the eyelet 16 comprises a base portion 18 having a top surface 22 and a bottom surface 20, and a post 24 protruding from the top surface 22 of the base portion 18. The bottom surface of the base portion of the eyelet 20 provides a surface area for contact with an electrolyte that is in direct contact with the skin of a patient. In alternate embodiments, the post 24 of the eyelet 16 may extend from the base 18 of the eyelet in other orientations. In additional alternate embodiments, the eyelet 16 can be configured without a post, in which case the eyelet contacts the snap by other means of connection.

The sensor or eyelet 16 is generally made from plastic and coated with silver/silver chloride on its bottom surface 20. The eyelet 16 is preferably made from a carbon-filled thermoplastic resin. The carbon fiber content of the resin is preferably in the range of about 10 percent to about 40 percent by weight, and more preferably, in the range of about 18 percent to about 22 percent by weight. In a particularly preferred embodiment, the eyelet 16 is made from high impact Acrylonitrile-Butadiene-Styrene (ABS) loaded with about 20 percent by weight of carbon fiber. The carbon fiber is preferably a polyacrylonitrile (PAN) based carbon fiber of a quality sufficient to assure the required electrical characteristics of the output material when molded.

The bottom surface of the eyelet 20 is coated with a conductive coating comprising silver chloride. The bottom surface of the eyelet 20 may be coated with silver and then treated to convert at least a portion of the silver to silver chloride. Preferably, the silver used is at least 99 percent pure fine silver. More preferably, the purity of the silver used is about 99.9% fine sliver. The conductive coating should be thick enough to provide sufficient conductivity for the desired application.

The conductive snap 34 is used as the terminal of the electrode. The snap 34 comprises a base portion 36 and a stud portion 38 extending from the base portion 36. The stud portion 38 of the snap 34 is preferably a hollow stud that can be press fit onto the post portion 24 of the eyelet 16. The hollow stud portion 38 has a sufficiently small inner diameter to snugly fit about the post portion 24 of the eyelet. The conductive snap 34 is generally made of a conductive metal, a metal alloy, or a conductive plastic resin. Commonly used materials include, for example, nickel plated brass, stainless steel, and carbon impregnated plastic.

The body of the electrode is comprised of a foam body 26. The body of the electrode can be made of any flexible substrate such as polyethylene foam, woven polyester fiber, or perforated tape. In a preferred embodiment, the body 26 is made of a foam material. The back surface 28 of the foam body 26 is coated with a biocompatible, pressure sensitive adhesive used to attach the electrode 10 to the patient's skin.

In order to provide a conductive pathway from the patient's skin to the electrode, an electrolyte layer 14 is placed over the silver/silver chloride coated bottom surface 20 of the eyelet 16. Preferably, the electrolyte layer 14 is slightly larger in dimension than the base portion 18 of the eyelet 16 so that no portion of the silver/silver chloride plated bottom surface 20 of the eyelet 16 is in contact with the patient's skin when the electrode 10 is used. The electrolyte layer 14 is generally a gel or viscous liquid. Commonly used gel materials for providing the conductive path from the skin to the bottom surface of the eyelet include hydrogel, adhesive gel, and liquid gel. Preferably the electrolyte 14 has low resistance/impedance and is capable of contouring to the skin of the patient. Generally, electrolytes contain about 2 percent to about 10 percent chloride salt as the conductor. In a preferred embodiment, hydrogel is used as the electrolyte 14. Hydrogel is a polymeric material which is conductive, preferably hydrophillic, has low surface resistivity, and good adhesive properties. It is most preferably hypoallergenic and includes a bacteriostat and fungistat. Such materials are well-known to those skilled in the art.

The protective release liner 12 is made of any conventional release liner material. Examples include silicone coated kraft paper or any plastic material which does not adhere strongly to the adhesive backing 28 on the body 26 of the electrode 10. In a preferred embodiment of the present invention, a layer of clear polyester plastic material is used as the release liner 12. Such release liner material is commercially available and is well-known to those skilled in the art. The release liner 12 preferably contains a tab 13 to facilitate removal of the release liner 12 before using the electrode 10.

In order to use the discretely coated electrode 10 of the present invention, the release liner 12 is peeled from the adhesive backing 28 of the foam body, revealing the adhesive backing 28 and the electrolyte layer 14. The electrolyte layer 14 remains stuck to the bottom surface 20 of the base portion 18 of the eyelet 16. The electrode 10 can then be pressed against the skin. The adhesive backing 28 serves to hold the electrode to the skin, and the electrolyte layer 14 provides electrical conductivity between the patient's skin and the electrode 10.

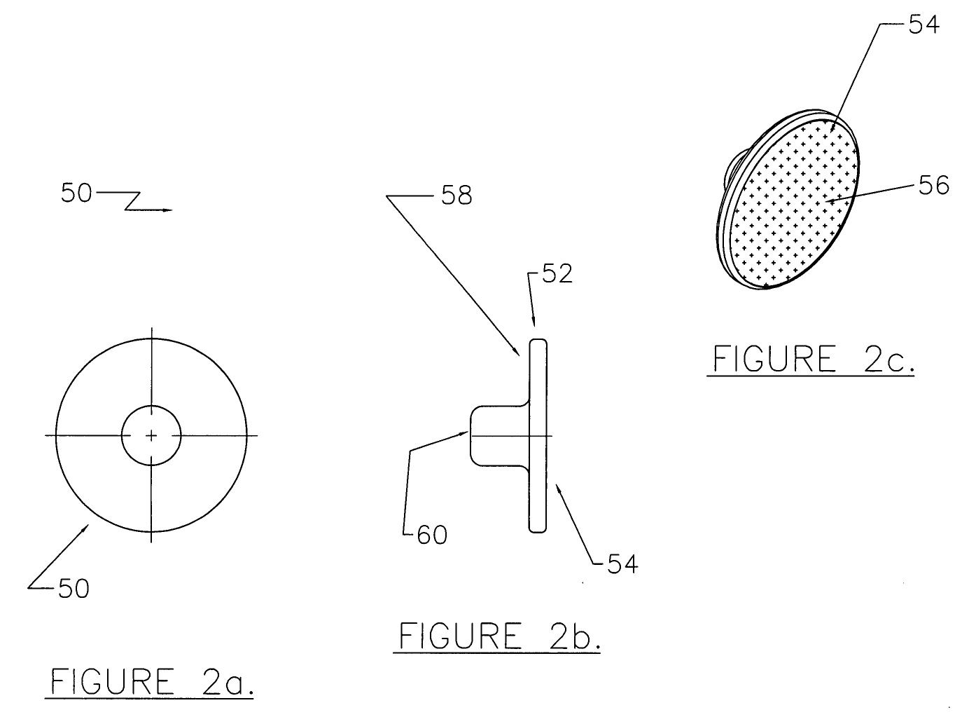

The discretely coated eyelet 50 is shown in greater detail in FIGS. 2a, 2b, and 2c. The eyelet 50 comprises a base portion 52 and a post portion 60 protruding from the base portion 52. The base portion of the eyelet 52 has a top surface 58 and a bottom surface 54. The bottom surface 54 of the base portion of the eyelet 50 provides a surface area which comes into contact with the electrolyte.

The sensor or eyelet 50 is generally made from a carbon filled thermoplastic resin and is coated with silver/silver chloride 56 on its bottom surface 54. In a preferred embodiment, the eyelet is made from high impact Acrylonitrile-Butadiene-Styrene (ABS) loaded with about 20 percent by weight of carbon fiber. The carbon fiber is preferably a polyacrylonitrile (PAN) based carbon fiber of a quality sufficient to assure the required electrical characteristics of the output material when molded. The bottom surface of the eyelet 54 is coated with a conductive metallic coating comprising silver chloride. The bottom surface of the eyelet 54 may be coated with silver and then treated to convert at least a portion of the silver to silver chloride. The silver used is preferably at least about 99 percent fine silver. More preferably, the silver used is about 99.9 percent fine silver. The conductive coating should be thick enough to provide sufficient conductivity for the desired application. Generally, the thickness of the conductive coating is within the range of about 50 microinches to about 200 microinches, but it is preferable to determine the desired thickness of the silver/silver chloride coating through the use of functional performance tests.

The eyelet 50 is generally manufactured by injection molding of a conductive resin. The conductive resin is preferably an ABS plastic resin impregnated with carbon fiber. Generally, the conductive resin includes about 10 to about 40 percent by weight of carbon fiber, and more preferably between about 18 and about 22 percent by weight of carbon fiber. In a particularly preferred embodiment, the eyelet is made of a conductive thermoplastic resin loaded with about 20 percent by weight of carbon fiber. The amount of carbon fiber is preferably sufficient to provide a resistance of about 50 ohms to about 100 ohms from the post portion 60 to the bottom surface 54 of the base portion 52 of the eyelet 50. The process is controlled to assure that dimensional characteristics specific to the final application and conductivity from the top of the post portion 60 to the bottom surface 54 of the base portion 52 are consistent.

Several different methods can be used to assure that the silver/silver chloride is applied only to the bottom surface 54 of the base portion 52 of the eyelet 50. For example, the parts of the eyelet 50 other than the bottom surface 54 of the base portion 52 of the eyelet 50 can be masked to prohibit exposure of these surfaces to the silver/silver chloride processing. Alternatively, the eyelet can be fixtured or positioned such that only the bottom surface 54 of the base portion 52 of the eyelet 50 is exposed to the silver/silver chloride processing.

There are various methods available for discretely coating the bottom surface 54 of the base 52 of the eyelet 50. Silver can be adhered or coated to the bottom surface 54 of the base 52 using silver metal, silver/silver chloride “ink”, silver solutions, or a combination of these materials.

The silver/silver chloride coating 56 can be applied by any method generally known in the art. In one embodiment, silver metal is used to coat the bottom surface 54 of the eyelet 50. The eyelet 50 can be masked or fixtured in order to present only the bottom surface 54 of the base portion 52 of the eyelet 50 for exposure to vacuum metallization. The eyelets are placed in a vacuum chamber, and pure silver is “sputtered” onto the exposed surface of the eyelet 50. After application of the silver, the silver surface is converted to silver/silver chloride using either an electrolytic or chemical process using chloride salts. Alternatively, the eyelets are placed in a vacuum chamber and a conductive “strike” layer is “sputtered” onto the exposed surface of the eyelet 50. After the “strike” layer is applied, the silver coating is then applied by “sputtering”, chemical deposition, or electroplating.

In another method utilizing silver metal, the eyelets are suitably prepared to present only the bottom surface 54 of the base 52 of the eyelet 50 for hot stamping or otherwise affixing a thin layer of silver foil onto the exposed surface. After application of the silver foil, the surface is converted to silver/silver chloride using either an electrolytic or chemical process using chloride salts.

In another embodiment, silver/silver chloride ink is used to coat the bottom surface 54 of the eyelet 50. The eyelets are suitably masked or fixtured to present only the bottom surface 54 of the eyelet 50 to a printing process. Silver/silver chloride ink is “painted” or “coated” onto the exposed surface using a gravure or pad-printing process, and the ink is then dried.

Alternatively, a thin conductive film (carbon impregnated) is coated with silver/silver chloride ink using a gravure printing process. The silver/silver chloride coating is dried. The conductive silver/silver chloride film is then “staked” to the bottom surface 54 of the eyelet 50 using a thermal or ultrasonic welding process. The conductive silver/silver chloride film is then trimmed to match the contour of the bottom surface 54 of the eyelet 50.

In another embodiment, silver solutions are used to coat the bottom surface 54 of the eyelet 50. The eyelets are suitably masked or fixtured such that only the bottom surface 54 of the eyelet 50 is exposed for further processing. The eyelets are subjected to an initial chemical deposition of silver as known in the art. Further chemical deposition may be employed to reach the final intended silver thickness. Eyelets may also be removed from the chemical deposition process and introduced to a silver electroplating process to increase the thickness of the silver. At the completion of the silver processing (either by chemical deposition or electroplating), the eyelets are then presented to a chemical or electrolytic process using chloride salts to convert the silver surface on the bottom surface 54 of the base portion 52 to a silver/silver chloride surface. A suitable percent of the silver is converted to silver chloride such that the component will meet the established industry requirements for the intended application.

In another embodiment, a base or “strike” coat of non-precious conductive metal is applied to the bottom surface 54 of the base portion 52 of the eyelet 50. After the “strike” layer is applied, the silver/silver chloride coating can be applied over the “strike” layer by either the chemical deposition or electroplating processes described above. Finally, the silver layer is converted to silver/silver chloride.

The silver/silver chloride conductive coating should be thick enough to provide sufficient conductivity. The thickness of the conductive coating is generally within the range of about 50 microinches to about 200 microinches. The desired thickness of the silver/silver chloride coating is preferably determined through the use of functional performance tests. There are standard minimum performance requirements and test methods for various types of electrodes provided by The American National Standards Institute (ANSI) and the Association for the Advancement of Medical Instrumentation (AAMI). For example, the ANSI/AAMI EC12 disposable ECG electrode standards are shown in the following table.

| Parameter | Standard |
|---|---|
| AC impedance | <2 kilohms (kO) average; |
| | no one pair >3 kilohms (kO) |
| DC offset voltage | <100 millivolts (mV) |
| Offset instability and | <150 microvolts (μV) for 5 minutes |
| internal noise | |
| Defibrillation overload | <100 millivolts (mV) after 5 seconds DC offset; |
| recovery | <1 millivolt/second (mV/sec) change in DC |
| | offset; |
| | <3 kilohms (kO) |
| Bias current tolerance | <100 millivolts (mV) for a minimum of 8 hours |

In the production of electrodes according to the present invention, the electrodes can be tested to assure that the required electrical parameters are met for the desired application, and the thickness of the silver/silver chloride coating can be adjusted accordingly. Alternatively, there may be a desired resistance for a given application. For example, in ECG electrodes it is generally preferred to provide a surface resistance of approximately 1 ohm per square inch. These types of functional tests can be used to determine the desired thickness of the silver/silver chloride coating.

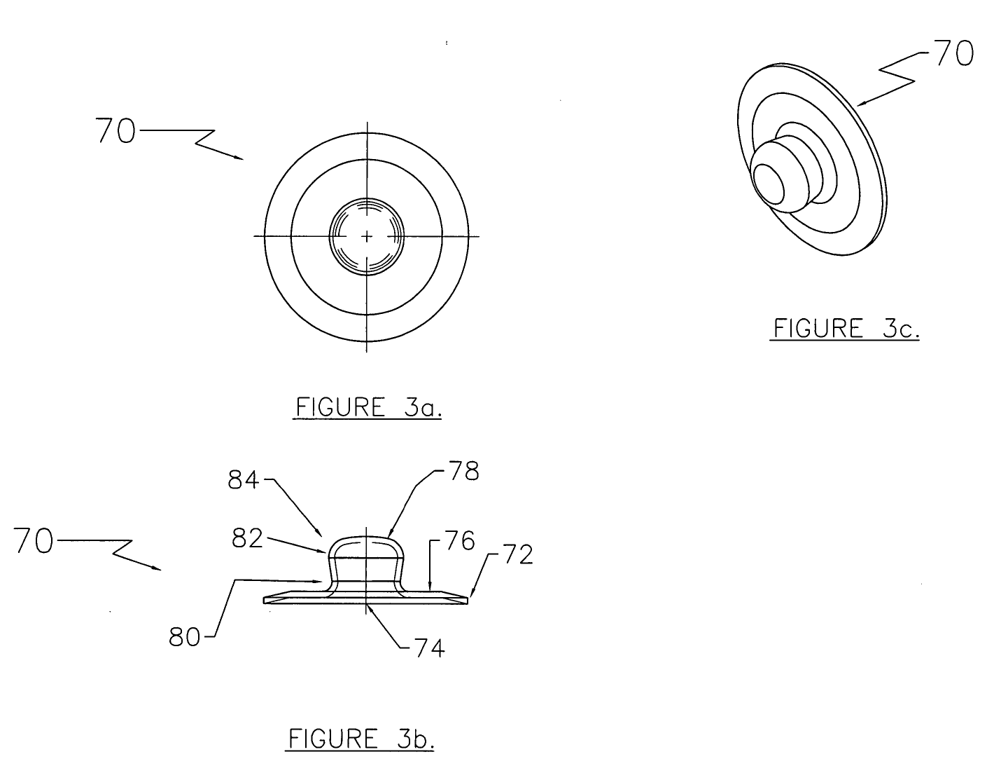

A representative conductive snap 70 is shown in greater detail in FIGS. 3a, 3b, and 3c. The conductive snap 70 is used as the terminal of the electrode. The snap 70 comprises a base portion 72 and a stud portion 78 that protrudes from the base portion 72. The stud portion 78 of the snap 70 is a hollow stud that can be press fit onto the post portion 60 of the eyelet 50. The hollow stud portion 78 has a sufficiently small inner diameter to create an interference fit about the post portion 60 of the eyelet 50. The conductive snap 70 is generally made of a conductive metal, a metal alloy, or a conductive plastic resin. Commonly used materials include, for example, nickel plated brass, stainless steel, and carbon impregnated plastic.

In a preferred embodiment of the present invention, the snap is made of brass. Due to the elimination of the dissimilar metal issue found in conventional silver/silver chloride electrodes, the electrodes of the present invention allow the use of more reactive metals, such as brass, in the snap 70 component.

The stud portion 78 of the snap 70 includes a top crown portion 84 and a bottom waist portion 80. The bottom waist portion 80 extends up from the base portion 72. The top crown portion 84 is preferably wider in circumference than the bottom waist portion 80 of the stud 78. With this configuration, the top crown portion 84 can be securely engaged into a conductive lead wire and a secure electrical connection can be made.

While one embodiment of a conductive snap is illustrated in the Figures, other configurations are also possible. An electrode system according to the present invention may utilize any type of conductive metal connector that can be mated to a discretely coated eyelet. Examples of connectors that can used include, but are not limited to, barrel connectors, pin adapters, and wires.

The present invention can also be applied to electrodes other than silver/silver chloride electrodes, although silver/silver chloride electrodes are most commonly used. The present invention can be applied to any discretely coated sensor for use in a medical electrode in which the connection between the snap and the sensor or eyelet does not result in the dissimilar metal phenomenon created in standard metal snap electrode designs. By coating the eyelet with a conductive metallic surface only on the area of the eyelet that is in contact with the electrolyte gel, the electrical performance and stability of the electrode are improved. For example, tin/stannous chloride (Sn/SnCl) electrodes can be produced so that the eyelet is coated with tin/stannous chloride only in the area in which the eyelet makes direct contact with the electrolyte gel in the conductive pathway. The remaining portions of the eyelet would remain substantially un-coated, so that the portion of the eyelet that contacts the conductive snap is preferably a conductive carbon loaded plastic material which is not coated and is not metallic.

It should be understood that various changes and modifications to the preferred embodiments described above will be apparent to those skilled in the art. For example, a discretely coated sensor according to the present invention can be used in any type of electrode system to eliminate the problems of having dissimilar metals in direct contact. Examples of electrode systems in which the present invention could be used include, but are not limited to, electrodes using barrel connectors, pin adaptors, and wire connections. These and other changes can be made without departing from the spirit and scope of the invention and without diminishing its attendant advantages. It is therefore intended that such changes and modifications be covered by the following claims.

1. A discretely coated eyelet for use in a medical electrode, the eyelet comprising:
a non-metallic, conductive material having a base portion, the base portion having a bottom surface and a top surface,
a post portion integral with and extending upwardly from the top surface of the base portion,
the bottom surface of the base portion of the eyelet being at least partially coated with a metallic conductive material, and the remaining surface of the base portion and the post portion of the eyelet being substantially un-coated.

2. The discretely coated eyelet according to claim 1 wherein the non-metallic, conductive material comprises a plastic resin loaded with carbon fiber.

3. The discretely coated eyelet according to claim 2 wherein the non-metallic, conductive material is loaded with about 10 percent to about 40 percent by weight of carbon fiber.

4. The discretely coated eyelet according to claim 2 wherein the non-metallic, conductive material is loaded with about 18 percent to about 22 percent by weight of carbon fiber.

5. The discretely coated eyelet according to claim 1 wherein the non-metallic, conductive material comprises Acrylonitrile-Butadiene-Styrene loaded with about 20 percent by weight of carbon fiber.

6. The discretely coated eyelet according to claim 1 wherein the metallic conductive material comprises at least one component selected from the group consisting of silver and a silver salt.

7. The discretely coated eyelet according to claim 1 wherein the metallic conductive material comprises silver/silver chloride.

8. The discretely coated eyelet according to claim 7 wherein the silver/silver chloride coating is applied using a printing-type process.

9. The discretely coated eyelet according to claim 1 wherein the metallic conductive material comprises a silver coating which is at least partially converted to silver chloride to produce a silver/silver chloride coating.

10. The discretely coated eyelet according to claim 9 wherein the silver coating is applied using a vacuum metallization process.

11. The discretely coated eyelet according to claim 9 wherein the silver coating is applied by affixing a thin layer of silver foil to bottom surface of the base portion of the eyelet.

12. The discretely coated eyelet according to claim 7 wherein the silver/silver chloride coating is about 50 microinches to about 200 microinches thick.

13. The discretely coated eyelet according to claim 7 wherein the silver/silver chloride coating is sufficiently thick to provide a surface resistance of about 1 ohm per square inch.

14. The discretely coated eyelet according to claim 4 wherein the metallic conductive material comprises at least one component selected from the group consisting of silver and a silver salt.

15. The discretely coated eyelet according to claim 4 wherein the metallic conductive material comprises silver/silver chloride.

16. The discretely coated eyelet according to claim 15 wherein the silver/silver chloride coating is applied using a printing-type process.

17. The discretely coated eyelet according to claim 4 wherein the metallic conductive material comprises a silver coating which is at least partially converted to silver chloride to produce a silver/silver chloride coating.

18. The discretely coated eyelet according to claim 17 wherein the silver coating is applied using a vacuum metallization process.

19. The discretely coated eyelet according to claim 17 wherein the silver coating is applied by affixing a thin layer of silver foil to bottom surface of the base portion of the eyelet.

20. The discretely coated eyelet according to claim 15 wherein the silver/silver chloride coating is about 50 microinches to about 200 microinches thick.

21. The discretely coated eyelet according to claim 15 wherein the silver/silver chloride coating is sufficiently thick to provide a surface resistance of about 1 ohm per square inch.

22. A discretely coated eyelet for use in a medical electrode, the eyelet comprising:
a non-metallic, conductive material having a base portion, the base portion having a bottom surface and a top surface,
a post portion integral with and extending upwardly from the top surface of the base portion,
the bottom surface of the base portion of the eyelet being at least partially coated with silver/silver chloride, and the remaining surface of the base portion and the post portion of the eyelet being substantially un-coated.

23. The discretely coated eyelet according to claim 22 wherein the non-metallic, conductive material comprises a plastic resin loaded with carbon fiber.

24. The discretely coated eyelet according to claim 23 wherein the non-metallic, conductive material is loaded with about 10 percent to about 40 percent by weight of carbon fiber.

25. The discretely coated eyelet according to claim 23 wherein the non-metallic, conductive material is loaded with about 18 percent to about 22 percent by weight of carbon fiber.

26. The discretely coated eyelet according to claim 23 wherein the non-metallic, conductive material comprises Acrylonitrile-Butadiene-Styrene loaded with about 20 percent by weight of carbon fiber.

27. The discretely coated eyelet according to claim 22 wherein the silver/silver chloride coating is about 50 microinches to about 200 microinches thick.

28. The discretely coated eyelet according to claim 22 wherein the silver/silver chloride coating is sufficiently thick to provide a surface resistance of about 1 ohm per square inch.

29. The discretely coated eyelet according to claim 25 wherein the silver/silver chloride coating is about 50 microinches to about 200 microinches thick.

30. The discretely coated eyelet according to claim 25 wherein the silver/silver chloride coating is sufficiently thick to provide a surface resistance of about 1 ohm per square inch.
